(19) United States
(12) Patent Application Publication
(10) Pub. No.: US 2022/0186164 A1
(43) Pub. Date: Jun. 16, 2022

(54) METHODS AND APPARATUSES FOR PROCESSING POULTRY LITTER

(71) Applicant: DVO Licensing Inc., Chilton, WI (US)

(72) Inventors: Doug VanOrnum, Chilton, WI (US); Stephen W. Dvorak, Chilton, WI (US)

(21) Appl. No.: 17/551,884

(22) Filed: Dec. 15, 2021

Related U.S. Application Data
(60) Provisional application No. 63/125,706, filed on Dec. 15, 2020.

(51) Int. Cl. C12M 1/107; C12M 1/00

(52) U.S. Cl. CPC C12M 21/04; C12M 47/10

(57) ABSTRACT

Methods, systems and apparatuses herein provide a unique and novel process to process poultry litter. In one embodiment, the poultry litter is anaerobically digested to produce a biogas and one or more nutrients. In one embodiment, the poultry litter is wettened with recycled digestate prior to anaerobic digestion.

### CROSS-REFERENCE TO RELATED APPLICATIONS

This application is a non-provisional application of and claims priority to Provisional Application No. 63/125,706 filed Dec. 15, 2020, which is incorporated herein by reference in its entirety.

### FIELD

Methods, systems, and apparatuses disclosed herein can be used to effectively and efficiently process poultry litter. In one embodiment, methods, systems, and apparatuses disclosed herein can be used to process poultry waste and generate biogas, and recover phosphorous and ammonia. Methods, systems, and apparatuses disclosed herein provide an integrated approach to waste management.

### BACKGROUND

Many million tons of poultry litter are produced annually in the United States virtually all from intensive systems. Poultry litter is solid waste material composed primarily of bedding material (any of a variety of lignocellulose materials), feathers, spilled animal feed, wood shavings, mortalities peanut hulls, and poultry excreta. The relative proportion of bedding to excreta can vary widely, as can the chemical nature of the litter. There also may be pathogens, weed seed, and drug contaminants present in the litter. Litter is, of course, malodorous due to various odorants or precursors thereof. In addition to free ammonia there have been identified odorants such as mercaptans, sulfides, di-ketones, indole, and skatole. Litter contains and during storage and composting generates many volatile organic compounds (VOCs).

Poultry litter is typically comprised of 30% bedding material and 70% excreta. As a result, the litter is a complex mixture of many compounds including sugars, fatty acids, cellulose, lignin and extractives, vitamins, and amino acids. Poultry litter naturally contains all of the nutrients, secondary nutrients, and micronutrients needed by plants including N, P, K, S, Zn, Ca, Mg, Mn, B, and Cu. The nutrient content of litter depends on many factors including management practices, the type of bedding material used, feeds, and more. Typically, on a dry basis poultry litter contains 1%-4% N, 25%-35% carbon, 1.4%-6.6% P₂O₅, 1.3%-4.1% K₂O and 0.3% to 2% S. Poultry litter also contains high levels of lignocellulose due to the bedding materials used in poultry houses. The bedding materials used are readily available forest and agricultural wastes such as straw, wood chips, peanut hulls, and rice hulls, for example. Poultry litter differs significantly from other wastes used to produce fertilizer. Poultry litter contains a multiplicity of organic compounds in addition to lignocellulose and these differ from organic compounds in manures, sewage, and biosolids.

Poultry litter is recognized as a serious source of nitrification of waters. As poultry production steadily grows due to demand for poultry products and population growth, so does the waste from this production. Efforts to protect our environment have led to regulations causing local producer/farmers to struggle with meeting state mandated nutrient management program requirements while remaining solvent with narrow profit margins.

Currently, the U.S. alone generates 13 million tons of poultry litter each year. The growing population consuming more poultry also needs an increasing food supply from crops, which require fertilizer. Poultry litter is used as fertilizer due to its well-documented source of primary plant nutrients (nitrogen, phosphorus, and potassium), secondary nutrients (sulfur, magnesium, and calcium), and micronutrients like zinc, copper, iron, boron, nickel, manganese, and molybdenum. However, using poultry litter as a fertilizer in either its raw form or after traditional treatments like composting or rotary drum heat treatment is not nutrient efficient, energy efficient, or safe to our health or environment. Also, even litter that has been heat treated will give off offensive odors when exposed to moisture or rain. Additionally, litter haulers are faced with a growing supply of poultry litter and a narrowing availability for land application. This leads to stockpiles of litter that further cause problems from release of greenhouse gases, potential leaching and run-off, human and animal exposure to pathogens, and loss of nutrients in the litter.

Poultry litter poses human health problems in various ways. Untreated litter dust not only smells bad but also carries pathogens in the air that can be dangerous to humans. These pathogens also have potential to be transmitted to livestock feeding on grass in fields treated with litter as well as to vegetables and other crops grown with litter used as fertilizer. Typical methods of mitigating this problem include composting or stacking the litter. These methods allow heat to kill the pathogens before applying.

Composting and stacking litter has an additional negative effect on our environment which is the release gases and reduced air quality. As stated above, pathogens can be transmitted to air by poultry litter. Composting litter also causes losses of both nitrogen and phosphorus due to denitrification and ammonia volatilization, run-off, and leaching. These losses can be quite high.

Anaerobic digestion (AD) of poultry manure has several important benefits: creates a more stable product for use, removes nuisance odors, maintains the nutrient value of the litter, reduces attraction for vectors and produces a renewable fuel. However, digesting poultry litter can be very operationally challenging, due to its heterogeneous and complex nature, which is one reason why more poultry-based AD systems are not in use today. Therefore, methods and apparatuses that can provide efficient and cost-effective anaerobic digestion of poultry litter are in great demand.

### BRIEF SUMMARY

The disclosure provides methods, systems, and apparatuses to convert poultry litter to numerous valuable commodities including but not limited to biogas, fertilizer, phosphorous, ammonia salts, and animal bedding.

In one embodiment, poultry litter is processed to produce a valuable dry, homogenous balanced granular or pelletized fertilizer free of noxious odors, free of harmful pathogens and viruses, free of viable weed seeds, and free of drugs, steroids, and pesticides.

In one embodiment, poultry litter is processed to produce animal bedding free of noxious odors, free of harmful pathogens and viruses, free of viable weed seeds, and free of drugs, steroids, and pesticides.

In one embodiment, the disclosure relates to a method for processing poultry litter comprising wetting poultry litter. In one embodiment, the poultry litter is wettened using digestate from an anaerobic digester. In one embodiment, the digestate has been removed from the digester and allowed to settle for a period of time. In one embodiment, the digestate is allowed to settle in a pit, a vat or a lagoon.

In one embodiment, the disclosure relates to a method for processing poultry litter comprising wetting poultry litter and removing biomass from the wettened poultry litter. In one embodiment, removing biomass from the wettened poultry litter comprises using a rotary drum.

In one embodiment, the disclosure relates to a method for processing poultry litter comprising wetting poultry litter and removing biomass from the wettened poultry litter without the addition of acid. In one embodiment, the disclosure relates to removing biomass from the wettened poultry litter without the addition of acid and does not require a partial neutralizing and ammoniating step.

In one embodiment, the disclosure relates to a method for processing poultry litter comprising: (a) wetting poultry litter; (b) separating biomass from the wettened poultry litter of step (a) to produce a poultry litter influent; and (c) digesting the poultry litter influent from step (b) in the anaerobic digester to produce an anaerobic digester effluent and a biogas.

In one embodiment, the disclosure relates to a method for processing poultry litter comprising: (a) wetting poultry litter to produce a wettened poultry litter, wherein total solids content of the wettened poultry litter is at least 50% less than the total solids content of the starting poultry litter; (b) separating biomass from the wettened poultry litter of step (a) to produce a poultry litter influent; and (c) digesting the poultry litter influent from step (b) in the anaerobic digester to produce an anaerobic digester effluent and a biogas.

In one embodiment, wetting poultry litter comprises using recycled digestate from an anaerobic digester. In another embodiment, separating biomass from the wettened poultry litter of step (a) comprises using a rotary drum.

In one embodiment, the disclosure relates to a method for processing poultry litter comprising: (a) obtaining a first anaerobic digester effluent from an anaerobic digester; (b) recovering phosphorous from the anaerobic digester effluent; (c) buffering and off-gassing the anaerobic digester effluent to produce a recycled digestate; (d) wetting poultry litter with recycled digestate; (e) separating woody biomass from the wettened poultry litter of step (d) to produce a poultry litter influent; and (f) digesting the poultry litter influent from step (e) in an anaerobic digester to produce a second anaerobic digester effluent and a biogas.

In one embodiment, the disclosure relates to a system comprising an anaerobic digester, a nutrient recovery system, a collection and wettening pit, a separation device, and a mixing tank.

In one embodiment, the disclosure relates to a system comprising an anaerobic digester configured to produce an anaerobic digester effluent; a nutrient recovery system configured to remove nutrients from the anaerobic digester effluent, including but not limited to ammonia and phosphorous, a settling system configured to allow buffering and off-gassing of the effluent and producing a recycled digestate; a collection and wettening pit to mix the recycled digestate with poultry litter; a separation device configured to remove woody biomass from the poultry litter and produce a poultry litter influent; and a mixing tank for mixing leachate from the woody biomass and the poultry litter influent. In one embodiment, the recycled digestate is also the wash water used in the separation device.

### BRIEF DESCRIPTION OF THE DRAWINGS

FIG. 1 is a representative schematic of a system for processing poultry litter comprising, inter alia, a pre-wetting pit, a rotary screen, a digester mix tank, an anaerobic digester and a nutrient recovery system.

FIG. 2 is a schematic of a representative embodiment of a system for processing poultry litter.

FIG. 3 is a schematic of one embodiment of a nutrient recovery system.

Before one embodiment of the invention is explained in detail, it is to be understood that the invention is not limited in its application to the details of construction and the arrangements of the components set forth in the following description or illustrated in the drawings. The methods and apparatuses are capable of other embodiments and of being practiced or being carried out in various ways. Also, it is understood that the phraseology and terminology used herein is for the purpose of description and should not be regarded as limiting. The use of “including” and “comprising” and variations thereof herein is meant to encompass the items listed thereafter and equivalents thereof as well as additional items.

### DETAILED DESCRIPTION

### Definitions

The numerical ranges in this disclosure are approximate, and thus may include values outside of the range unless otherwise indicated. Numerical ranges include all values from and including the lower and the upper values, in increments of one unit, provided that there is a separation of at least two units between any lower value and any higher value. As an example, if a compositional, physical or other property, such as, for example, molecular weight, viscosity, melt index, etc., is from 100 to 1,000, it is intended that all individual values, such as 100, 101, 102, etc., and sub ranges, such as 100 to 144, 155 to 170, 197 to 200, etc., are expressly enumerated. For ranges containing values which are less than one or containing fractional numbers greater than one (e.g., 1.1, 1.5, etc.), one unit is considered to be 0.0001, 0.001, 0.01 or 0.1, as appropriate. For ranges containing single digit numbers less than ten (e.g., 1 to 5), one unit is typically considered to be 0.1. These are only examples of what is specifically intended, and all possible combinations of numerical values between the lowest value and the highest value enumerated, are to be considered to be expressly stated in this disclosure. Numerical ranges are provided within this disclosure for, among other things, relative amounts of components in a mixture, and various temperature and other parameter ranges recited in the methods.

As used herein, the singular forms “a,” “an,” and “the” refer to one or more than one, unless the context clearly dictates otherwise.

As used herein, the term “anaerobic digester effluent” includes effluent directly removed from the anaerobic digester, effluent removed from the digester and separated from large solids, effluent removed from the digester and separated from fine solids, effluent removed from the digester and separated from large and fine solids; effluent removed from the digester and aerated; effluent removed from the digester and heated; effluent removed from the digester and heated and aerated; effluent removed from the digester heated and aerated and separated from solids; effluent removed from the digester heated, aerated and used to remove H₂S from a biogas; effluent removed from the digester heated, aerated, separated from solids, and used to remove H₂S from biogas; effluent removed from the digester heated, aerated, used to remove H₂S from a biogas and regenerated to an alkaline pH; effluent removed from the digester heated, aerated, separated from solids, used to remove H₂S from biogas, and regenerated to an alkaline pH; effluent removed from the digester heated, aerated, used to remove H₂S from a biogas, regenerated to an alkaline pH, and used to remove CO₂ from a biogas; and effluent removed from the digester heated, aerated, separated from solids, used to remove H₂S from biogas, regenerated to an alkaline pH and used to remove CO₂ from biogas.

In one embodiment, anaerobic digester effluent comprises effluent obtained from digesting waste material in an anerobic digester having a hydraulic retention time of 21 days.

In one embodiment, anerobic digester effluent comprises effluent obtained from an anerobic digester at the conclusion of digestion of the waste material.

As used herein, the terms “bioreactor,” “reactor,” or “fermentation bioreactor,” include a fermentation device consisting of one or more vessels and/or towers or piping arrangement, which includes the Continuous Stirred Tank Reactor (CSTR), Immobilized Cell Reactor (ICR), Trickle Bed Reactor (TBR), Bubble Column, Gas lift Fermenter, Static Mixer, or other device suitable for gas-liquid contact.

As used herein, the term “chicken manure” is intended to refer to chicken excreta that can be used as a fertilizer.

As used herein, the term “includes” means “comprises.” For example, a device that includes or comprises “A” and “B” contains “A” and “B” but may optionally contain “C” or other components other than “A” and “B.” A device that includes or comprises “A” or “B” may contain “A” or “B” or “A” and “B,” and optionally one or more other components such as “C.”

As used herein, the term “layered manure” refers to an unadulterated waste product from egg laying chickens.

As used herein, the term “manure” refers to animal wastes including animal dejections, feed remains and hair.

As used herein, the term “manure slurry” is intended to refer to a mixture of manure and any liquid, e.g., urine and/or water. Thus, in one aspect, a manure slurry can be formed when animal manure and urine are contacted, or when manure is mixed with water from an external source. No specific moisture and/or solids content is intended to be implied by the term slurry.

As used herein, “poultry litter” is a heterogeneous mix of manure, urine, bedding, waste feed, feathers and mortalities, with common bedding materials including sawdust, shavings, wheat straw, peanut hulls, and rice hulls.

As used herein, the term “quicklime” is calcium oxide (CaO). Quicklime is a white, caustic and alkaline crystalline solid at room temperature. As a commercial product, lime often also contains magnesium oxide, silicon oxide and smaller amounts of aluminum oxide and iron oxide.

As used herein, the term “removing or reducing CO2” refers to eliminating an amount or percentage of CO₂ in biogas. The percentage eliminated can be as small as 0.5% or greater than 200%.

As used herein, the term “removing or reducing H₂S” refers to eliminating an amount or percentage of H₂S in biogas. The percentage eliminated can be as small as 0.5% or greater than 200%.

As used herein, the term “treated biogas” refers to biogas that has been in direct or indirect contact with an alkaline effluent.

As used herein, the term “recycled digestate” refers to effluent obtained after a nutrient recovery process followed by a period of buffering and off-gassing. In one embodiment, recycled digestate comprises effluent obtained after a nutrient recovery process to remove phosphorous from anaerobic digester effluent followed by a period of buffering and off-gassing. In one embodiment, recycled digestate comprises effluent obtained after a nutrient recovery process to remove ammonia from anaerobic digester effluent followed by a period of buffering and off-gassing.

### Waste Processing System

As briefly described above, the present disclosure relates to methods for treating animal manure and waste products from, for example, poultry and livestock production facilities.

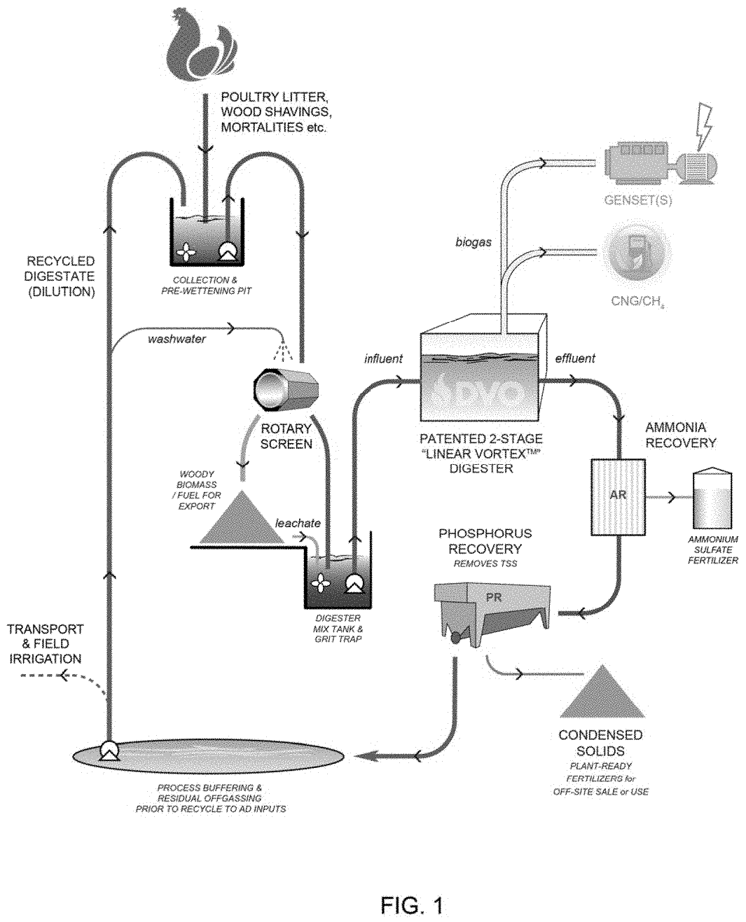

In one embodiment, a waste processing system is illustrated in FIG. 1. In one representative, non-limiting embodiment, the waste processing system is used to process poultry litter. The system 10 includes a wetting pit/tank (20), a rotary screen (30), a digester mix tank (40), and an anerobic digester (50). In some embodiments, the system (10) can further include a nutrient recovery system (60). The system 10 can be used to process waste material and remove contaminants, including H₂S, from biogas, thereby producing a biogas that can be used for electricity and as a fuel source.

The nutrient recovery system includes solid separation for phosphorous removal, NH₃ removal via air stripping and acid absorption with an appropriate acid, such as sulfuric acid (H₂SO₄). The heat required in the NH₃ process can be recovered from the CHP engine.

As can be seen from the schematic in FIG. 1, at the end of the nutrient recovery process, the effluent is at a pH of around 9.7, which should be reduced before storage in a lagoon or applied as a fertilizer. The effluent can be recycled (recycled digestate 70) and mixed with poultry litter in the wetting pit.

### Waste Material

Poultry and other livestock are commonly reared in facilities that are designed to manage manure and liquid waste generated by such animals. For example, poultry are typically raised on beds of litter that contain a filler such as wood shavings, wood chips and/or saw dust, spilled food, feathers, and manure. After a growout on the bed of litter and during successive growouts, the litter is predominantly manure, and is eventually replaced with fresh bedding.

In addition to livestock production, farmers raise poultry for the production of eggs. Through industry advances, farmers now raise these animals in cages in buildings reaching as high as six stories. The large amount of manure produced is often accumulated and stored in outdoor holding areas.

Farmers manage the manure and liquid waste from livestock rearing facilities in several ways. For example, many farmers apply the manure and liquid waste onto agricultural fields. Other farmers spread the manure and liquid waste from the facilities directly onto their land.

Manure excreted by poultry and other livestock typically contains a variety of pathogens, including Salmonella, Coliform, Fecal Coliform, Soil Transmitted Helminths (hookworm, Ascaris, and whipworm), Campylobacter, Avian Influenza, Histoplasma, Capsulatum Fungus, and Escherichia coli. The presence of these pathogens poses health risks to farm workers handling the manure. In addition, the use or distribution of manure containing these pathogens on agricultural crops can pose health and environmental concerns to farm workers and consumers.

In various aspects, the methods of the present disclosure can utilize and/or treat animal manure from a variety of animals, such as, for example, poultry. In one aspect, the animal waste stream to be treated can comprise poultry manure. In other aspects, the waste stream can comprise animal waste, feces, urine, food, bedding materials, such as wood chips and/or sawdust, feathers, and other materials. In another aspect, a poultry litter can contain one or more harmful microorganisms, such as bacteria, viruses, protozoa, and/or other parasites or pathogens.

Animal waste can be provided from an on-site facility or can be delivered, for example, in bulk quantities by truck. It should also be understood that the properties, for example, the nutrient content and physical properties of a given animal waste product can vary depending upon, for example, the type of animal and/or rearing or growth facility, length of time the animal waste has been stored, environmental conditions, etc. In one aspect, properties, such as, for example, nitrogen content, phosphorus content, potassium content, calcium content, sulfur content, boron content, magnesium content, molybdenum content, sodium content, manganese content, zinc content, iron content, copper content, moisture, and pH, can vary depending upon the type of animal and/or rearing or growth facility. For example, poultry litter animal waste can contain woodchips, sawdust, feathers, and/or other materials in addition to feces, and the moisture content can vary depending upon whether the litter originated in a broiler or egg-laying facility. Poultry litter can comprises a variety of materials of varying size.

The conventional methods to handle manure and liquid waste products from poultry and livestock production facilities do not address the health and environmental concerns described herein. Thus, the abundance of animal manure, such as chicken manure, and the problems associated with its disposal led to the development of a new process for manufacturing organic liquid and solid fertilizers by aerobic decomposition of animal manure, as described herein.

Waste material may be collected using any suitable means in the art. Waste material includes but is not limited to wood, grass, agricultural residue, manure, recycled waste paper, and agricultural waste materials. Examples of sources of waste materials include, but are not limited to, livestock production facilities, such as cattle, swine, goat, sheep, dairy cow, horse and the like, chicken ranches, turkey farms, duck farms, geese farms, human waste, and the like. Waste material may also include many forms of agricultural products processing facilities that may include non-food related agricultural products. Waste material may also include some forms of commingled wastes where a portion of the waste may also include food scraps. Waste material also may include commingled fibers with spoiled foods.

In another embodiment, the waste material also may include hay, straw, and other material commonly used in animal stalls or other agriculture environment. In yet another embodiment, the waste material also may contain urine plus water used in cleaning the stalls. In still yet another embodiment, the waste material may also contain additional material, such as twine, rope, and other material that may or may not be biodegradable. In yet another embodiment, the waste material is from a dairy farm.

In another embodiment, the waste material also may include fibers from non-food agricultural products such as bamboo, oil palm, coir, etc.

In one non-limiting embodiment, the waste processing system disclosed herein is used to process poultry litter.

### Wetting Pit

The waste processing system 10 comprises a wetting pit 20 for mixing poultry litter 5 with anaerobic digester effluent, including but not limited to recycled digestate 70. The poultry litter is wettened to allow thorough mixing in the anaerobic digester. In one embodiment, the moisture content of the poultry litter can be increased by at least two-fold as compared to the starting poultry litter material. In another embodiment, the moisture content of the poultry litter can be increased by at least four-fold, or at least eight-fold, or at least ten-fold as compared to the moisture content of the starting poultry litter material.

In one embodiment, the total solids content of the wettened poultry litter is from 50-99% less of the total solids content of the starting poultry litter, or from 60-99% less of the total solids content of the starting poultry litter, or from 70-99% less of the total solids content of the starting poultry litter, or from 80-99% less of the total solids content of the starting poultry litter, or from or from 90-99% less of the total solids content of the starting poultry litter.

In one embodiment, the total solids content of the wettened poultry litter is reduced by 30%, or 40%, or 50%, or 60%, or 70%, or 80%, or 90%, or, 91, or 92, or 93, or 94, or 95%, or 96%, or 97%, or 98%, or 99% as compared to the total solids content of the starting poultry litter.

In one embodiment, the poultry litter remains in a wetting pit or enclosure for less than 48 hours. In one embodiment, poultry litter remains in a wetting pit or enclosure from 1 hour to 24 hours. In one embodiment, poultry litter remains in a wetting pit or enclosure from 6 hours to 24 hours. In one embodiment, poultry litter remains in a wetting pit or enclosure from 12 hours to 24 hours. In one embodiment, poultry litter remains in a wetting pit or enclosure from 18 hours to 24 hours.

In one aspect, the moisture content can be adjusted, if needed, at this or any subsequent step of the process, using water and/or a nutrient enriched liquid, including but not limited to recycled digestate 70.

In one aspect, use of a nutrient enriched liquid can minimize and/or eliminate dilution of one or more desirable nutrients that can be present in the animal waste. Exemplary nutrients can include those compounds beneficial for fertilizer or agricultural applications, such as, nitrogen, phosphorus, and potassium. In one aspect, a nutrient enriched liquid can be water derived from the treatment methods described herein, for example, water that has been in contact with animal waste, including but not limited to recycled digestate 70.

In another aspect, a nutrient enriched liquid can be separately prepared using animal waste or desirable chemical compounds. In one aspect, the nutrient enriched liquid is prepared from water contacted with animal waste, which does not introduce non-organic components into the treatment process. The proportions of nutrient enriched liquid and water, for example, clean or municipal water, used in the treatment process can vary, depending upon the particular animal waste product being treated and/or the desired properties of the resulting treated product. In various aspects, the proportion can range from 100% water to 100% nutrient enriched liquid, and the present disclosure is intended to include all combinations there between.

In one embodiment, the wetting pit can have any desired shape or dimensions that achieve the desired result including but not limited to a rectangle, a square, hexagonal, octagonal, round, a circle, a triangle, a pentagon and a V-notched shape.

### Separation Device

In one embodiment, the waste processing system comprise a separation device to separate woody biomass from the liquid. In one embodiment, the separation device may vary depending on the precise poultry bedding materials. Rice hulls, wood shavings, sawdust, peanut hulls, shredded sugar cane, crushed corn cobs, processed paper, chopped straw are some, but not all, examples of material used for poultry bedding. In some situations, materials are combined to produce the poultry bedding. Representative, non-limiting examples of separation devices include but are not limited to a mechanical separator, an inclined auger, a slopescreen (also called a sidehill), a screwpress, a centrifuge, a perforated conveyor, and a rotary screen.

### A. Rotary Screen

The wettened poultry litter is introduced within the interior of a rotary screen 30. Washwater, which can be recycled digestate 70, is then turned so as to cause the poultry litter to enter the cylinder of the rotary screen. At the same time, the cylinder of the rotary screen is revolved by means of the gearwheels. In this way, the waste material under treatment is supported by the portion of the rotary screen, which happens for the time being to be lowermost. When so supported, it is elevated slightly and then allowed to drop back of its own accord, so that it is constantly turned over—and over again.

While being thus turned over and mixed, it is subjected to the action of the washwater, which can, as a result of the continuous movement of the matter, penetrate and act upon every portion thereof. As a result, the wash-water acts thoroughly and effectively and quickly. The woody biomass falls through the perforated screen and collects at the lowermost portion of the cylinder. The remaining liquid/influent can be transported to a mixing tank. In one embodiment, the liquid/influent is transported through the use of one or more pipes.

### Mixing Tank

In one embodiment, the liquid/influent from the separation device is mixed with leachate 105 from the woody biomass in a mixing tank. The leachate/influent is then transported to an anaerobic digester.

In one embodiment, the leachate is from about 5% to about 25% of the leachate/effluent composition. In one embodiment, the leachate is from 1% to 5% of the leachate/effluent composition. In one embodiment, the leachate is from 0.1 to 1% of the leachate/effluent composition. In one embodiment, the leachate is from 0.01% to 1% of the leachate/effluent composition.

In one embodiment, the influent from the separation device is from 60% to 85% of the leachate/influent composition. In one embodiment, the influent from the separation device is from 80% to 99% of the leachate/influent composition.

### Anaerobic Digester

Influent from the mixing tank is transported to an anaerobic digester (50). In one embodiment, the influent is transported from the mixing tank to an anaerobic digester via one or more pipes.

Any type of anaerobic digester may be used. A conventional anaerobic digester system generally includes the following components: manure transfer and mixing pit, a digester made of steel, fiberglass, concrete, earth or other suitable material (including heating and mixing equipment if needed), biogas handling and transmission, and gas end use (combustion) equipment such as electric generation equipment.

Conventional anaerobic digesters can also require significant operational oversight depending on operational mode and temperature. Conventional anaerobic digester systems also require proper design and sizing to maintain critical bacterial populations responsible for waste treatment and stabilization for sustained long-term predictable performance. Sizing requirements are based on hydraulic retention time (HRT), and loading rate, where the operating temperature affects these sizing parameters. These factors (size, materials, operational requirements) affect digester costs, which may be fairly capital intensive, and in some economies and farm scales, may not be affordable or may be inoperable if experienced technicians are not available.

In one embodiment, anaerobic digesters having any type of process configuration can be used including but not limited to batch, continuous, mesophilic temperature, thermophilic temperature, high solids, low solids, single-stage complexity and multistage complexity.

In another embodiment, a batch system of anaerobic digestion can be used. Biomass is added to the reactor at the start of the process in a batch and is sealed for the duration of the process. Batch reactors suffer from odor issues that can be a severe problem when they are emptied. Typically biogas production will be formed with a normal distribution pattern over time. The operator can use this fact to determine when they believe the process of digestion of the organic matter has completed.

In yet another embodiment, a continuous system of anaerobic digestion can be used. In continuous digestion processes, organic matter is typically added to the reactor in stages. The end products are constantly or periodically removed, resulting in constant production of biogas. Examples of this form of anaerobic digestion include, continuous stirred-tank reactors (CSTRs), Upflow anaerobic sludge blanket (UASB), Expanded granular sludge bed (EGSB) and Internal circulation reactors (IC).

In still another embodiment, mesophilic or thermophilic operational temperature levels for anaerobic digesters can be used. Mesophilic temperature levels take place optimally around 37°-41° C. or at ambient temperatures between 20°-45° C.; under these temperatures, mesophiles are the primary microorganism present. Thermophilic temperature levels take place optimally around 50°-52° C. and at elevated temperatures up to 70° C.; under these temperatures, thermophiles are the primary microorganisms present.

There are a greater number of species of mesophiles than thermophiles. Mesophiles are also more tolerant to changes in environmental conditions than thermophiles. Mesophilic systems are therefore considered to be more stable than thermophilic digestion systems.

In another embodiment, anaerobic digesters can either be designed to operate in a dry-solids, not liquefied content, with a total suspended solids (TSS) concentration greater than 20%, or a low solids concentration with a TSS concentration less than 15%. High-solids digesters process a thick slurry that requires more energy input to move and process the feedstock. The thickness of the material may also lead to associated problems with abrasion. High-solids digesters will typically have a lower land requirement due to the lower volumes associated with the moisture.

Low-solids (high solids, liquefied) digesters can transport material through the system using standard pumps that require significantly lower energy input. Low-solids digesters require a larger amount of land than high-solids due to the increased volumes associated with the increased liquid: feedstock ratio of the digesters. There are benefits associated with operation in a liquid environment as it enables more thorough circulation of materials and contact between the bacteria and food. This enables the bacteria to more readily access the substances they are feeding off and increases the speed of gas yields.

In still another embodiment, digestion systems can be configured with different levels of complexity: one-stage or single-stage and two-stage or multistage. A single-stage digestion system is one in which all of the biological reactions occur within a single sealed reactor or holding tank. Utilizing a single-stage reactor reduces the cost of construction; however there is less control of the reactions occurring within the system. For instance, acidogenic bacteria, through the production of acids, reduce the pH of the tank, while methanogenic bacteria operate in a strictly defined pH range. Therefore, the biological reactions of the different species in a single-stage reactor can be in direct competition with each other. Another one-stage reaction system is an anaerobic lagoon. These lagoons are pond-like earthen basins used for the treatment and long-term storage of manures. In this case, the anaerobic reactions are contained within the natural anaerobic sludge contained in the pool.

In a two-stage or multi-stage digestion system, different digestion vessels are optimized to bring maximum control over the bacterial communities living within the digesters. Acidogenic bacteria produce organic acids and grow and reproduce faster than methanogenic bacteria. Methanogenic bacteria require stable pH and temperature in order to optimize their performance.

The residence time in a digester varies with the amount and type of waste material, the configuration of the digestion system and whether it is one-stage or two-stage. In the case of single-stage thermophilic digestion residence times may be in the region of 14 days, which comparatively to mesophilic digestion is relatively fast. The plug-flow nature of some of these systems will mean that the full degradation of the material may not have been realized in this timescale. In this event, digestate exiting the system will be darker in color and will typically have more odor associated with it.

In two-stage mesophilic digestion, residence time may vary between 15 and 40 days. In the case of mesophilic UASB digestion, hydraulic residence times can be (1 hour-1 day) and solid retention times can be up to 90 days. In this manner, the UASB system is able to separate solid and hydraulic retention times with the utilization of a sludge blanket.

Continuous digesters have mechanical or hydraulic devices, depending on the level of solids in the material, to mix the contents enabling the bacteria and the food to be in contact. They also allow excess material to be continuously extracted to maintain a reasonably constant volume within the digestion tanks.

In one embodiment, the waste material can be processed through an anaerobic digester available from DVO, Inc. (Chilton, Wis.). In one embodiment, the waste material can be processed through an anaerobic digester as described in any of U.S. Pat. Nos. 6,451,589; 6,613,562; 7,078,229; and 7,179,642; each of which are incorporated by reference in their entirety. Each of the patents recited above is assigned to GHD, Inc., which is now DVO, Inc., and names Mr. Steve Dvorak as the sole inventor. In yet another embodiment, the anaerobic digester can be a two-stage mixed plug flow digester system

In another aspect, the disclosure may provide a method for the anaerobic digestion of high-solids waste comprising moving the solid waste in a corkscrew-like fashion through the digester. The digester is a generally U-shaped tank with overall horizontal dimensions of approximately 100 feet long and 72 feet wide. A center wall approximately 90 feet in length divides the digester into the two legs of the U-shape. Thus, each leg of the digester is approximately 100 feet long and 36 feet wide.

Modified plug flow or slurry flow can be used to move the sludge. The digester heating pipes locally heat the sludge using hot water at approximately 160° F. from the cooler of the engine, causing the heated mixed sludge to rise under convective forces. The convection develops a current in the digester that is uncharacteristic of other digesters. Sludge is heated by the digester heating pipes near the digester center wall, such that convective forces cause the heated sludge to rise near the center wall. At the same time, sludge near the relatively cooler outer wall falls under convective forces. As a result, the convective forces cause the sludge to follow a circular flow path upward along the center wall and downward along the outer wall. At the same time, the sludge flows along the first and second legs of the digester, resulting in a combined corkscrew-like flow path for the sludge.

In another embodiment (not shown), hot gas injection jets using heated gases from the output of the engine replace the hot water digester heating pipes as a heating and current-generating source. The injection of hot gases circulates the sludge through both natural and forced convection. A similar corkscrew-like flow path is developed in the digester.

To further increase upward flow of the heated sludge near the center wall, biogas may be removed from the biogas storage area in the digester, pressurized with a gas centrifugal or rotary-lobe blower, and injected into the heated sludge through nozzles positioned onto conduit. This recycled biogas injection near the floor of the digester serves to increase the rapidity of the cork-screw-like flow path for the heated sludge.

The U-shape of the digester results in a long sludge flow path and thus a long residence time of approximately twenty days. As the sludge flows through the digester, anaerobic digestion processes the sludge into activated sludge. From the digester, the activated sludge flows into the optional clarifier and into effluent pit 110. The clarifier uses gravity to separate the activated sludge into liquid and solid portions.

### Nutrient Recovery System

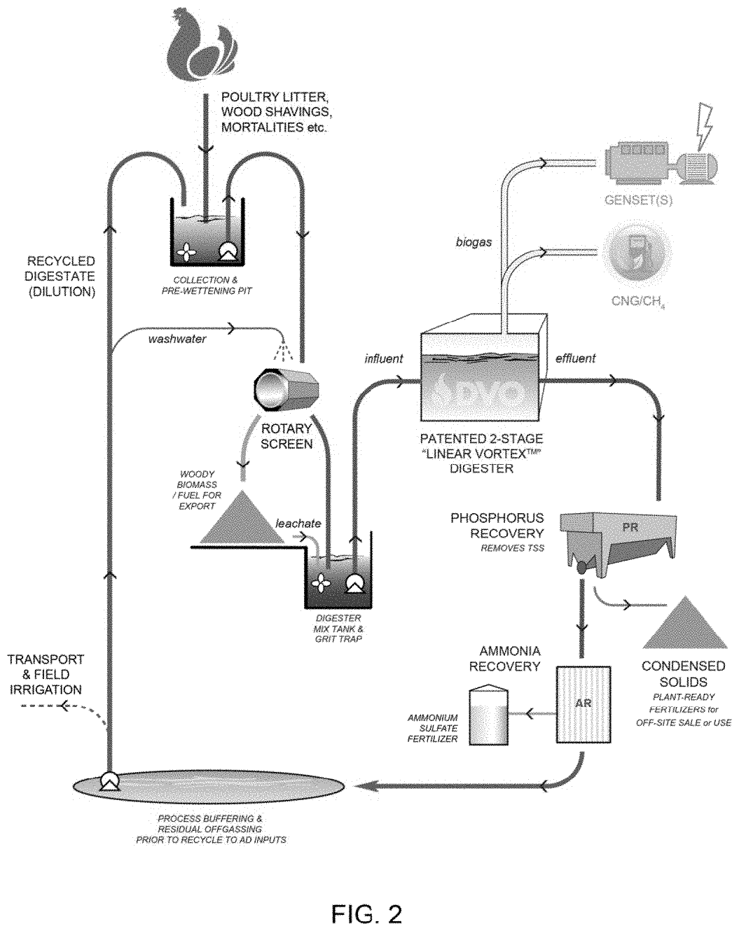

In one embodiment, a nutrient recovery system 200 is illustrated in FIG. 2. In one embodiment, the effluent from the anaerobic digester may gravity flow, or it can be pumped, into an insulated effluent pit 110. In an embodiment, the anaerobic digester effluent is discharged from the digester, while maintaining gas integrity. The discharge of the anaerobic digester effluent is designed to maximize turbulence, thin film flow, and contact with outside air. This discharge process results in degassing of supersaturated methane gas for greater gas production and environmental/climate control.

In an embodiment, the resulting methane/air mixture can be re-injected into the anaerobic digester for enhancing mixing, and increasing biogas production. In addition, the re-injected methane/air mixture can aid in reducing hydrogen sulfide content in the digester.

The temperature of the effluent may be raised as it flows through the first vessel in a plug flow process to a suitable temperature including but not limited to 100° F. to 110° F., 110° F. to 120° F., 120° F. to 130° F., 130° F. to 140° F., 140° F. to 150° F., 150° F. to 160° F., 160° F. to 165° F., 165° F. to 175° F., and 175° F. to 195° F.

In an embodiment, the effluent is heated using an extended exhaust heat recovery system to further heat treat the effluent and its fibrous solids to Class A pathogen standards.

The hydraulic retention time (HRT) of the influent in the vessel can be verified according to U.S. EPA standards. HRT may vary, depending on design criteria, from 30 minutes to 48 hours or from 4 hours to 36 hours or from 8 hours to 24 hours or from 12 hours to 16 hours.

The effluent pit 110 will have a gas headspace above the liquid level and below the vessel ceiling, will be air tight, and will be operated under a vacuum. The effluent in the effluent pit will be heated and agitated by the injection of heated gas, including but not limited to air, through injectors or gas nozzles 120. The heated gas will be injected into the liquid near the bottom of the effluent pit, causing a corkscrew mixing effect. Heated air can be supplied by taking ambient air through a cross-flow heat exchanger 122, with the exhaust from the bio-gas engine generator set providing the heated air stream. Heated effluent, agitated with air, will release the majority of the CO₂ and some of the NH₃ entrained in the liquid waste. Releasing the CO₂ from the liquid waste will cause a rise in pH in the liquid waste, increasing the NH₃ removal efficiency. The pH value can be used as a marker for how much supersaturated gas has been released. The pH value also can be used as a marker to determine what nutrients can be recovered.

Not to be bound by any particular theory, it is believe that aeration allows for the stripping of super-saturated gases including but not limited to CO₂, and that high temperature enhances the kinetics, according to Henry's law, allowing for a more rapid release the supersaturated gases. By aerating the effluent, the pH value is increased and gases, which may interfere with natural flocculation and settling are removed from the liquid effluent.

Additionally, the release of supersaturated gases is tied to the important shifts in chemical equilibria that occur as result of aeration and Henry's Law, such as shifts in carbonate, bicarbonate, and ammonia system equilibria. Aeration allows for the release of supersaturated CO2 but also results in a decrease in total inorganic carbon (carbonates, bicarbonates, etc), which also occurs and leads to more gas release and continued change in pH.

In an embodiment, the aeration rate can be any rate that achieves or assists in the release of supersaturated gases including but not limited to from 2 gallons/cfm to 160 gallons/cfm, or from 5 gallons/cfm to 150 gallons/cfm, or from 10 gallons/cfm to 100 gallons/cfm or from 25 gallons/cfm to 80 gallons/cfm or from 40 gallons/cfm to 50 gallons/cfm. In an embodiment, micro-aeration socks can be used.

In an embodiment, the aeration time can be any amount of time that achieves the release of supersaturated gases including but not limited to from 15 min to 3 days, or from 2 hours to 2 days, or from 4 hours to 24 hours, or from 8 hours to 18 hours, or from 12 hours to 16 hours.

In an embodiment, the aeration rate is selected to allow for stripping of supersaturated gases and maintaining the level of existing struvite or struvite-like colloidal solids. In an embodiment, the aeration rate does not cause or limit dissolution of struvite-like particles, which would release more free phosphates.

In an embodiment, aeration can increase the pH value of the effluent to a desired value including but not limited to 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, 10.1, 10.2, 10.3, 10.4, 10.5, 10.6, 10.7, 10.8, 10.9, 11.0, 11.1, 11.2, 11.3, 11.4, 11.5, 11.6, 11.7, 11.8, 11.9, 12.0 and greater than 12.0. The heating and aeration and the subsequent pH increase effectively eliminates pathogens from the liquid effluent, producing a sterilized liquid.

In another embodiment, the aeration source is designed to produce bubbles of a particular size including but not limited bubbles produced through microaeration, macroaeration, or through the use of different sparger configurations, types, sizes and shapes.

In yet another embodiment, the method comprises prior to aeration of the effluent, separating digested material from the effluent. In yet another embodiment, the method comprises prior to aeration of the effluent, separating digested fibrous material from effluent.

In another embodiment, separating digested material from effluent can be achieved through the use of mechanical methods including but not limited to screens, filters, and column separation.

In one embodiment, the effluent can have large and fine solids. In another embodiment, the effluent can have only fine solids. In still another embodiment, the effluent can have only large solids. In still yet another embodiment, the effluent can comprise in relation to the total solid content in the effluent from 1 to 5% large solids, or from 5 to 10% large solids, or from 10 to 15% large solids, or from 15 to 20% large solids, or from 20 to 25% large solids, or from 25 to 30% large solids, or from 30 to 35% large solids, or from 35 to 40% large solids, or from 40 to 45% large solids, or from 45 to 50% large solids, or from 50 to 55% large solids, or from 55 to 60% large solids, or from 60 to 65% large solids, or from 65 to 70% large solids, or from 70 to 75% large solids, or from 75 to 80% large solids, or from 80 to 85% large solids, or from 85 to 90% large solids, or from 90 to 95% large solids, or from 95 to 99% large solids.

In still yet another embodiment, the effluent can comprise in relation to the total solid content in the effluent from 1 to 5% fine solids, or from 5 to 10% fine solids, or from 10 to 15% fine solids, or from 15 to 20% fine solids, or from 20 to 25% fine solids, or from 25 to 30% fine solids, or from 30 to 35% fine solids, or from 35 to 40% fine solids, or from 40 to 45% fine solids, or from 45 to 50% fine solids, or from 50 to 55% fine solids, or from 55 to 60% fine solids, or from 60 to 65% fine solids, or from 65 to 70% fine solids, or from 70 to 75% fine solids, or from 75 to 80% fine solids, or from 80 to 85% fine solids, or from 85 to 90% fine solids, or from 90 to 95% fine solids, or from 95 to 99% fine solids.

### Stripping Tower

The nutrient recovery system also comprises a stripping tower (140). The stripping tower is used for absorbing gaseous ammonia and stabilizing it to ammonium salt solution, which can be more concentrated and easily stored. Briefly, stripping is a distillation procedure that consists of separating fluid components by differences in boiling point or vapor pressure. The usual means of separation is through a column or tower that is packed with one or more various support materials, i.e. Pall Rings, Raschig Rings, Berl Saddles, etc., to increase contact surface. A stripping medium (e.g. hot air or steam, or, in one embodiment, unheated air) is injected into the bottom of the tower and an ammonia containing solution is injected at or near the top. As the ammonia containing liquid trickles down through the packing, it contacts the rising hot vapor and the more volatile ammonia fraction is vaporized and can be collected and further treated. The less volatile liquid component becomes increasingly purer as it nears the bottom of the tower, where it may be collected. U.S. Pat. No. 7,909,995, which issued on Mar. 22, 2011, provides additional information on designs of stripping towers and nutrient recovery systems, and is expressly incorporated herein by reference in its entirety.

The stripping tower is an apparatus that can hold caustic acids including but not limited to sulfuric acid, nitric acid, carbonic acid, hydrochloric acid, and phosphate acid. The stripping towers can also comprise vacuum blowers and pumps.

In one embodiment, the stripping tower can be used to collect any ammonium salt including but not limited to ammonium carbonate, ammonium sulfate, ammonium chloride, ammonium nitrate, and ammonium phosphate.

As opposed to conventional methods that flow manure through stripping towers, plug flow aeration can be employed. This avoids clogging concerns that plaque stripping towers. In addition, conventional stripping towers focus on high efficiency through very high aeration rates. These aeration rates are often associated with pressure drops and high electricity demands.

In one embodiment, ammonia stripping is carried out using a closed loop tower design that uses air as the stripping medium and includes an acid absorption system to capture ammonia as ammonium salt. Air can be used for this process because, although it does not have as high an ammonia absorbance capacity as other potential carrier gases, air is inexpensive and the pH adjustment needed can be maintained at a relatively low level (e.g. pH 10) because the process takes advantage of the hot (about 32-35° C.) manure wastewater coming from the anaerobic digester to compensate.

In one embodiment, a single tower design may be used. A single tower includes waste water input for ammonia stripping and acid input for acid absorption. Air is directed into the bottom of the tower using the fan or blower. Air circulates in an enclosed system, thus allowing for enhanced ammonia recovery and a reduction in energy inputs as the air without outside influence maintains its temperature for a longer period of time. In some embodiments, the air is heated, e.g. to a temperature of about 50° C., or in the range of from about 40° C. to about 60° C. In another embodiment, a two tower system can be used.

In an embodiment, the effluent air in the effluent pit 110 will be transferred to a packed stripping tower 140 where a liquid wash of sulfuric acid (or other acid) will drop the pH of the liquid stream (combined liquid wash and effluent air) and create a solution comprising ammonium sulfate. The solution can comprise an ammonium-salt slurry comprising from about 30% to about 60% ammonium sulfate. The ammonium sulfate can be collected and used as fertilizer. In another embodiment, other acids and contact chemicals can be used to produce any number of ammonium salts including but not limited to ammonium nitrate, ammonium phosphate, ammonium citrate, each which can serve as a fertilizer.

In one embodiment, the effluent air, under vacuum, in the effluent pit 110 will be transferred to a packed stripping tower 140.

### Solid/Liquid Separator

At the end of the engineered HRT, the sterilized effluent will be pumped to a solids/liquid separator 130; resulting in a separated solids 135 stream that will meet Class A bio-solids criteria and a separator liquid stream 137 that will also be sterilized and pathogen free. In another embodiment, at the end of the engineered HRT, the effluent will be strongly reduced in pathogens.

The separated solids and separated liquid will be reduced in ammonia-N content. The ammonium sulfate created will be a higher-value utilization of the natural ammonium found in organic wastes and will be in a chemical form that is easier to utilize and market. The separated solids can be utilized for animal bedding, horticultural usage, or fertilizer. The removal and capture of ammonia from the liquid effluent also reduces the natural release of ammonia gas into the atmosphere from waste storage and disposal and thus, reduces nox, N₂O and greenhouse gas emissions and the environmental effects associated with ammonia release and these other nitrogen gas releases to the atmosphere.

### Air-Tight Vessel

The separator liquid stream, temperature maintained from 130° F. to 180° F. or from 140° F. to 160° F. can be transferred to a single chamber or multi-chamber air-tight vessel. A three chamber air-tight vessel 145 is shown in FIG. 2. The first chamber 150 is separated from the second chamber 160 by a barrier wall. The second chamber 160 is separated from the third chamber 170 by a barrier wall.

In an embodiment, the barrier wall can be made of any suitable material that keeps the chambers distinct including but not limited to plastic PVC, polyethylene, polypropylene, methacrylic or acrylic plastic, fiber glass reinforced plastic (FRP), or stainless steel.

In an embodiment, the first and third chambers can be in any shape or dimension that allows the desired outcome including but not limited to a rectangle, a square, a triangle, a circle, a pentagon and a V-notched shape. One or more pumps can be located at or near the floor of the first and/or third chambers.

### a. The First Chamber

The first chamber 150, which may not be utilized in all configurations, will be a “quiet zone” chamber where the separator liquid will be allowed to decant. The large percentage of the minute solids that passed through the solids separator with the liquid effluent likely will settle to the bottom of the first chamber 150 and will be collected and removed for dewatering. Anaerobic digested and aerated liquids with decreased solids content, due to a separation process, and at a higher liquid temperature, separate faster and more efficiently. The liquid stream will plug flow through the first chamber 150, designed with an HRT from 30 minutes to 24 hours or from 60 minutes to 18 hours or from 2 hours to 16 hours or from 4 hours to 12 hours or from 8 hours to 10 hours. The liquid stream will plug flow into the second chamber 160.

### b. The Second Chamber

The second chamber 160 can have any desired shape or dimensions that achieve the desired result including but not limited to a rectangle, a square, a circle, a triangle, a pentagon and a V-notched shape.

In the second chamber 160, the liquid stream may be gas-agitated with air that is heated in a heat exchanger with the engine exhaust. Nozzles or jets for injection of air into the second chamber can be located at or near the floor of the second chamber 160. In another embodiment, the liquid stream may be hydraulic-agitated with a recirculation pump, or can be mechanically agitated with a prop agitation system. In an embodiment, the agitation can be for a suitable period of time including but not limited to 30 minutes to 1 hour, 1 hour to 2 hours, 2 hours to 4 hours, 4 hours to 6 hours, 6 hours to 8 hours, 8 hours to 10 hours, 10 hours to 12 hours and greater than 12 hours.

In an embodiment, the liquid stream will have continuous agitation, which will aid in the removal of ammonia if removal is desired.

In one embodiment, a high pH liquid including but not limited to quicklime or a caustic, can be added to the separated liquid stream, upon entering the second chamber, to increase the pH of the liquid effluent to a suitable value including but not limited to 9.0-9.1, 9.1-9.2, 9.2-9.3, 9.3-9.4, 9.4-9.5, 9.5-9.6, 9.6-9.7, 9.7-9.8, 9.8-9.9, 9.9-10.0, 10.0-11.0, 11.0-12.0, 12.0-12.5, and greater than 12.5.

A benefit of decreasing the solids content of a waste liquid is that less lime or caustic or in the case of no caustic addition, less aeration time and rate, is needed to raise the pH of a given volume of liquid, thereby decreasing the chemical treatment cost of the nutrient recovery system. The liquid stream will plug flow through the second chamber 160 of the air-tight vessel 140 as it is agitated utilizing the mixed plug flow (corkscrew) agitation method described above in the section entitled Anaerobic Digesters, and will thereby maintain a consistent HRT in the vessel.

Increasing the pH of an anaerobic digester effluent to a pH of about 9.5 or higher, at a temperature from 110° F. to about 160° F. or greater, will convert soluble ammonium-nitrogen (NH₄—N) to non-soluble, volatile ammonia nitrogen (NH₃—N). The ammonia-nitrogen 162 will be volatilized rapidly with the continuous agitation provided in the air tight vessel and will be collected in the head space provided in the vessel. Vacuum extraction of the head space gases may be utilized to further increase the volatilization rate inside the air tight vessel. Subsequently, by utilizing a system of air scrubbing the gaseous air stream with a low pH liquid solution of H₂SO₄ or similar acidic chemical, in a cross-flow air stripping tower 140, the ammonia will be removed from the air stream and captured as liquid ammonium sulfate. Ammonium sulfate is a highly valuable, easily solid fertilizer utilized by farmers and it will be an income stream for the nutrient removal system. Most importantly, the removal of the ammonium-nitrogen from the liquid waste stream solves one of the major disposal issues of the anaerobic digester effluent: high nitrogen content. In addition, removal of the ammonium-nitrogen also limits the natural discharge of NH₃ and N₂O into the atmosphere.

### c. Third Chamber

The liquid stream will plug flow into a third chamber 170, a “quiet zone” with no agitation where the liquid will be allowed to decant. The remaining solids will settle to the bottom of the third chamber, where they can be removed by a bottom discharge separation system. By the addition of quicklime, with its high pH and magnesium component, and the high temperature agitation that preceded the third chamber, a high level of magnesium-ammonium-phosphate (struvite) easily and readily settles. The settled solids will be removed from the third chamber 160 and dewatered.

In an embodiment, settling and dewatering of the nutrient rich solids is made easier through the use of a primary pump. In another embodiment, acid can be added to condense the solids layer for decanting.

Magnesium-ammonium-phosphate is also a highly valuable, easily sold fertilizer utilized by farmers and it will also be an income stream for the nutrient removal system. By removing the phosphorus and more ammonium from the liquid waste stream, the two largest disposal issues of the anaerobic digester effluent have been removed. The methods, systems and apparatuses disclosed herein contribute to solving many of the environmental and regulatory issues that generators/disposers of liquid organic wastes encounter in the US.

### Heat Exchanger

The decanted liquid with a temperature from 140° F. to 175° F. will be pumped to a waste-to-waste heat exchanger 180 where the temperature from the decanted liquid will be conserved by heating the cool incoming raw organic wastes at the front of the anaerobic digester system 10. This will conserve heat costs in the total system.

The decanted liquid will proceed from the heat exchanger 180 to a cross-flow, packed tower gas scrubbing system 190. In this gas scrubbing tower 190, the high pH decanted liquid will be exposed to the biogas 200 from the anaerobic digester system 10. The anaerobic digester biogas 200 typically has a hydrogen sulfate (H₂S) content of 500 ppm or higher and is considered very corrosive to the reciprocating engines utilized to convert the biogas into power for the electrical generation process.

The reaction in the stripping tower 190 of the high pH decanted liquids with the acidic H₂S found in the biogas stream lowers the H₂S level in the biogas to less than 50 ppm. This lower H₂S concentration in the biogas and significantly reduces the operation and maintenance costs of the reciprocating engines in the AD system.

Additionally, the high pH of the decanted liquid is now lowered to approximately 8.0 after neutralizing the acidic H₂S and removing a significant percentage of CO₂ from the biogas; resulting in a more friendly-to-use liquid for the farmer/owner and easier liquid disposal options. Other options such as biogas bubbling chamber and micro-diffusers can also be utilized in lieu of a stripping tower.

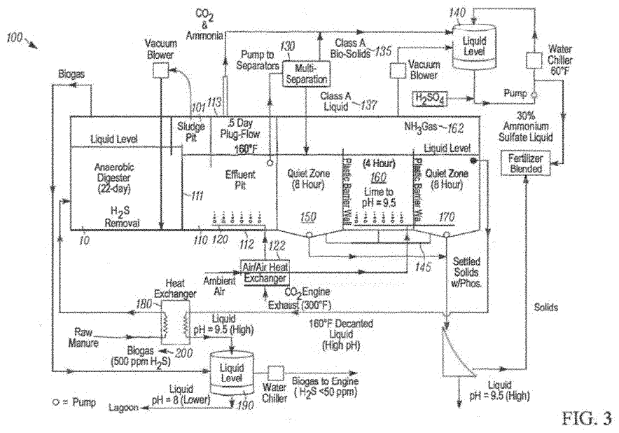

FIG. 3 shows another embodiment of a nutrient recovery system 300. Nutrient recovery system 300 is similar to system 100, with the exception that a two-chamber air tight vessel 310 is shown.

The nutrient recovery system 300 comprises an effluent pit 110 that comprises a heat exchanger 315 to heat the anaerobic digester effluent. The effluent pit also comprises a pump to transport the anaerobic digester effluent into the first chamber 320 of the two-chamber air-tight vessel 310.

The two-chamber air tight vessel 310 has a chamber 320 that allows for the liquid stream to be gas-agitated with air that is heated in a heat exchanger 322 with the engine exhaust. Nozzles or jets 324 for injection of air into chamber 320 can be located at or near the floor of chamber 320. In another embodiment, the liquid stream may be hydraulic-agitated with a recirculation pump, or can be mechanically agitated with a prop agitation system. In an embodiment, the agitation can be for a suitable period of time including but not limited to 30 minutes to 1 hour, 1 hour to 2 hours, 2 hours to 4 hours, 4 hours to 6 hours, 6 hours to 8 hours, 8 hours to 10 hours, 10 hours to 12 hours and greater than 12 hours

In an embodiment, the effluent is adjusted to a pH value ranging from 9.0 to 10.5. In an embodiment, a pH value of greater than 9.5 can be achieved by aeration, or aeration and the addition of an agent with a high pH value including by not limited to a caustic or quicklime. The addition of an agent with a high pH value can be used to increase the pH to a value of 9.5-10.0, 10.0-10.5, 10.5-11.0, 11.0-11.5, 11.5-12.0, 12.0-12.5, and greater than 12.5.

The heated and high pH effluent can be pumped to a multi-separator 130 that separates solids 135 from liquids 137, which satisfy the requirements for the liquids and solids to be considered Class A. The liquid effluent is pumped into chamber 340, which is a quite zone. The remaining components, recovery processes, and pH adjustments of the liquid effluent are essentially as described for system 100.

In one embodiment, the disclosure relates to a system comprising an anaerobic digester, a nutrient recovery system, a buffering system, a collection and wettening pit, a separation device, and a mixing tank.

In one embodiment, the disclosure relates to a system comprising an anaerobic digester configured to produce an anaerobic digester effluent; a nutrient recovery system configured to remove nutrients from the anaerobic digester effluent, including but not limited to ammonia and phosphorous, a settling system configured to allow buffering and off-gassing of the effluent and producing a recycled digestate; a collection and wettening pit to mix the recycled digestate with poultry litter; a separation device configured to remove woody biomass from the poultry litter and produce a poultry litter influent; and a mixing tank for mixing leachate from the woody biomass and the poultry litter influent. In one embodiment, the recycled digestate is also the wash water used in the separation device.

The invention is now described with reference to the following Examples. These Examples are provided for the purpose of illustration only and the invention should in no way be construed as being limited to these Examples, but rather should be construed to encompass any and all variations that become evident as a result of the teaching provided herein. All references including but not limited to U.S. patents, allowed U.S. patent applications, or published U.S. patent applications are incorporated within this specification by reference in their entirety.

What is claimed is:

1. A method for processing poultry litter comprising:
(a) wetting poultry litter with recycled digestate;
(b) separating woody biomass from the wettened poultry litter of step (a) to produce a poultry litter influent;
(c) digesting the poultry litter influent from step (b) in an anaerobic digester to produce an anaerobic digester effluent and a biogas.

2. The method of claim 1, wherein the recycled digestate is digestate obtained after a nutrient recovery process to remove phosphorous.

3. The method of claim 1, wherein the recycled digestate is digestate obtained after a nutrient recovery process to remove ammonia.

4. The method of claim 1, wherein removing woody biomass comprises using a rotary screen.

5. The method of claim 1, wherein the anaerobic digester employs a mixed plug flow design.

6. The method of claim 1, wherein the anaerobic digester uses a cork-screw flow path to move the waste fibrous material through the digester.

7. The method of claim 1, further comprising prior to step (c) mixing the poultry litter influent in a mixing tank with leachate from the separated woody biomass.

8. The method of claim 1, further comprising step (d): processing the anaerobic digester effluent to recover one or more nutrients.

9. The method of claim 8, wherein the anaerobic digester effluent is processed to recover phosphorous.

10. The method of claim 8, wherein the anaerobic digester effluent is processed to recover ammonia.

11. The method of claim 8, wherein processing the anaerobic digester effluent to recover one or more nutrients comprises:
(i) heating and aerating anaerobic digester effluent in an aeration reactor to convert soluble ammonium to gaseous ammonia;
(ii) providing gaseous ammonia from the aeration reactor to a stripping tower, said stripping tower providing controlled amounts of acid that reacts with gaseous ammonia; and
(iii) recovering an ammonium salt produced from reacting the acid with gaseous ammonia in the stripping tower.

12. The method of claim 11, further comprising pumping the anaerobic digester effluent from the aeration reactor to a solids settling system after providing the gaseous ammonia to the stripping tower.

13. The method of claim 12 further comprising collecting phosphorous-rich solids from the solids settling system.

14. A method for processing poultry litter comprising:
(a) obtaining a first anaerobic digester effluent from an anaerobic digester;
(b) recovering one or more nutrients from the anaerobic digester effluent;
(c) buffering and off-gassing the anaerobic digester effluent of step (b) to produce a recycled digestate;
(d) wetting poultry litter with recycled digestate of step (c);
(e) separating woody biomass from the wettened poultry litter of step (d) to produce a poultry litter influent; and
(f) digesting the poultry litter influent from step (e) in an anaerobic digester to produce a second anaerobic digester effluent and a biogas.

15. The method of claim 14, wherein recovering one or more nutrients comprises recovering phosphorous from the anaerobic digester effluent.

16. The method of claim 15 wherein recovering one or more nutrients further comprises recovering ammonia from the anaerobic digester effluent.

17. The method of claim 14, further comprising, prior to step (f), mixing the poultry litter influent in a mixing tank with leachate from the separated woody biomass.

18. A system for processing poultry litter comprising
(a) an anaerobic digester configured to produce an anaerobic digester effluent;
(b) a nutrient recovery system configured to recover one or more nutrients from the anaerobic digester effluent of step (a);
(c) a settling system configured to allow buffering and off-gassing of the effluent from step (b), thereby producing a recycled digestate;
(d) a collection and wettening pit to mix the recycled digestate of step (c) with poultry litter;
(e) a separation device configured to remove woody biomass from the poultry litter and produce a poultry litter influent; and
(f) a mixing tank for mixing leachate from the woody biomass of step (e) and the poultry litter influent.

19. The system of claim 18, wherein the separation device is a rotary drum.

20. The system of claim 19, wherein the rotary drum uses recycled digestate from step (c) to wash the poultry litter.
